(19) United States
(12) Patent Application Publication
(10) Pub. No.: US 2009/0269424 A1
(43) Pub. Date: Oct. 29, 2009

(54) Peripheral blood flow-improving composition

(75) Inventors: Hirofumi Koda, Osaka (JP); Yoshinoby Kiso, Osaka (JP); Takehiro Matsumoto, Osaka (JP)

Correspondence Address: DRINKER BIDDLE & REATH (DC), 1500 K STREET, N.W., SUITE 1100, WASHINGTON, DC, 20005-1209, US

(73) Assignee: SUNTORY HOLDINGS LIMITED

(21) Appl. No.: 12/458,157

(22) Filed: Jul. 1, 2009

Related U.S. Application Data
(63) Continuation of application No. 10/594,905, filed on Aug. 1, 2007.

(30) Foreign Application Priority Data
Mar. 31, 2004 (JP) JP-2004-101735

(51) Int. Cl. A61K 36/00

(52) U.S. Cl. 424/725

(57) ABSTRACT

The present invention provides a composition that can effectively improve peripheral blood flow to thereby ameliorate cold sensation, numbness, pains and other skin symptoms, as well as frostbites, chilblains and chaps and other skin disorders associated with disturbed peripheral blood flow. This composition has no side effects. Specifically, the peripheral blood flow-improving composition contains, as an active ingredient, a extract of a plant of the genus Lepidium of the family Cruciferae. More specifically, the composition contains as an active ingredient a extract of maca plant (Lepidium meyenii Walp). Food and beverage products, perfumes and cosmetics, and pharmaceutical products containing the peripheral blood flow-improving composition are also provided.

### TECHNICAL FIELD

The present invention relates to compositions for improving peripheral blood flow, and in particular, to a peripheral blood flow-improving composition that contains extracts of plants of the genus Cruciferae, in particular, maca plant. The present invention also relates to food and beverage products that contain the peripheral blood flow-improving composition.

### BACKGROUND ART

Conventional drugs to ameliorate cold sensation, numbness and pains associated with disturbed peripheral blood flow act both by dilating blood vessels and by preventing platelet coagulation. Not only are the therapeutic effects of these drugs less than satisfactory, but these drugs also act on blood vessels throughout the body and are thus accompanied by headaches, flush and many other side effects. These drugs are particularly unsafe when used with people of advanced age.

As the population ages, a growing number of patients are suffering from cold sensation, numbness and pains caused by the disturbed peripheral blood flow. There is thus an increasing demand for the development of drugs that can ameliorate these symptoms by acting locally on the affected site. Such drugs are expected to have less systemic effects and are safe for use with people of advanced age.

Many common skin problems, such as frostbites, chilblains and chaps, are caused by the disturbed peripheral blood flow. Other skin disorders, such as bacterial infection, may also be aggravated by the defective flow of peripheral blood.

Oil cosmetics and external medications containing blood flow-promoting agents are generally used in the prevention and treatment of these skin disorders. These external skin medications do not achieve sufficient improvement of peripheral blood flow and are less than satisfactory. Thus, there is a need for the development of drugs that are safe and can ameliorate frostbites, chilblains, chaps and other skin disorders associated with the disturbed peripheral blood flow.

Maca (Lepidium meyenii Walp) is a plant of the genus cruciferae (brassicaceae) that is originally grown in highlands of Peruvian Andes. The plant grows as it crawls on the ground surface and has roots that resemble a radish. Historically, maca has been cultivated in the Andes region for about two thousand years and has been eaten for the purpose of promoting health. It primarily contains polysaccharides and proteins and is also a rich source of various amino acids, including essential amino acids, which cannot be synthesized in our body and must therefore be taken from food. Vitamins (e.g., vitamin B, C and E) and minerals (e.g., calcium, iron and zinc) are also abundant in maca plants. In Peru, maca is much appreciated health-promoting diet and is used in a wide variety of food products, ranging from cookies and a beverage known as “CHICHA DE MACA” to maca liquors and powdery sprinkles on yoghurt.

It has been long known that maca brings about revitalizing and energizing effects. A composition that contains maca to provide anticancer effects and promote sexual function has been described (Patent Document 1). Another composition contains maca in combination with deer antler in the hope of increasing testosterone level in humans (Patent Document 2). It has also been suggested that maca has a potential use as anti-inflammatory or anti-allergy agents (Non-Patent Document 1). Furthermore, an external medication has been proposed that uses maca extracts to improve skin whitening and keep the skin moist (Patent Document 3).

Patent Document 1: U.S. Pat. No. 6,267,995 B1 Publication
Patent Document 2: Published Japanese Translation of PCT application No. 2003-523945

### Patent Document 3: Japanese Patent Laid-Open Publication No. 2001-39854 Non-Patent Document 1: IGAKU TO SEIBUTSUGAKU (Medicine and Biology), vol. 145, No. 6, p. 81-86 (2002)

As described, the various advantageous effects of maca have drawn much attention and new food and beverage products have come out that use maca in the form of dried and crushed product or extracts. In the course of our studies about the effects of maca, the present inventors have found that maca extracts have the ability to effectively improve the peripheral blood flow. It is this discovery that led to the present invention.

No previous studies have been conducted to investigate the ability of maca extracts to improve peripheral blood flow. In this regard, the present invention is highly unique and unprecedented.

### DISCLOSURE OF THE INVENTION

### Problems to be Solved by the Invention

Accordingly, it is an object of the present invention to provide a composition that has no side effects and can effectively improve peripheral blood flow to thereby ameliorate cold sensation, numbness, pains and skin disorders associated with the disturbed peripheral blood flow.

### Means for Solving the Problems

One essential aspect of the present invention concerns a composition for improving peripheral blood flow, containing, as an active ingredient, an extract of a plant of the genus Lepidium of the family Cruciferae.

More specifically, the present invention is the peripheral blood flow-improving composition, wherein the plant of the genus Lepidium of the family Cruciferae is maca (Lepidium meyenii Walp).

Thus, one most essential aspect of the present invention concerns the peripheral blood flow-improving composition containing a maca extract as an active ingredient.

More specifically, the present invention is the peripheral blood flow-improving composition, wherein the maca extract is obtained by adding an aqueous ethanol solution to a crushed maca product, and by maintaining the mixture at 40 to 85° C. for extraction.

Another aspect of the present invention concerns a food and beverage product, perfume and liquor, and pharmaceutical product that contain the above-described peripheral blood flow-improving composition.

### EFFECT OF THE INVENTION

According to the present invention, there is provided a peripheral blood flow-improving composition that contains maca extracts. Symptoms such as cold sensation, numbness and pains, as well as skin disorders, such as frostbites, chilblains and chaps caused by disturbed peripheral blood flow are often associated with dry skin. Although several approaches, including external cosmetics and ointments and bath agents, have been proposed to prevent dry skin, none of them have provided a decisive solution. The peripheral blood flow-improving composition provided by the present invention also serves as an effective approach to the problem of dry skin without giving any side effects.

### BEST MODE FOR CARRYING OUT THE INVENTION

As described above, the present invention provides a peripheral blood flow-improving composition that contains extracts of plants of the genus Lepidium of the family Cruciferae. A preferred plant of the genus Lepidium of the family Cruciferae is maca (Lepidium meyenii Walp).

Maca is a root vegetable cultivated or naturally growing in highlands of Peruvian Andes at altitudes of 4000 to 5000 m. The plant's roots of maca have been eaten since ancient time, by local peoples.

The present invention will now be described with reference to maca (Lepidium meyenii Walp) as a representative plant of the genus Lepidium of the family Cruciferae.

The extracts of plants of the genus Lepidium of the family Cruciferae for use in the present invention can be obtained from any of the plant's parts, including whole maca plants, flowers, fruits, leaves, stems and subterranean stems and bulbs. Of the different plant's parts, bulbs are preferably used in the extraction process using an extraction solvent. When desired, the plant materials are dried, crushed, or cut for extraction.

The extraction solvent may be any solvent, including water, organic solvents and mixtures thereof.

Specific examples include lower alcohols, such as methanol, ethanol and butanol; polyols, such as propylene glycol and 1,3-butylene glycol; esters, such as ethyl acetate and amyl acetate; and ketones, such as acetone and methyl ethyl ketone.

Because the extracts may be intended for oral ingestion according to the present invention, mixtures of water and ethanol (i.e., aqueous ethanol solutions) are preferred to ensure safety.

The studies conducted by the present inventors have revealed that the amount of ethanol in the aqueous ethanol solution used as the extraction solvent affects the ability of the extracts to improve the peripheral blood flow. Thus, it is important to adjust the ratio of ethanol to water in the aqueous ethanol solution to optimize the effect of the extracts. Specifically, the amount of ethanol is from 30% to 100% by volume, and in particular from about 40% to about 99% by volume.

It is also important to adjust the temperature of the extraction solvent during the extraction process to optimize the effect of the extracts. To facilitate the process, the extraction is preferably carried out at lower temperatures than the boiling point of the solvent. Specifically, the temperature of the solvent is maintained at 40 to 100° C., and in particular at about 60 to about 85° C. during the extraction process.

Upon extraction, maca and the solvent may be mixed in any suitable proportions: it is preferred that about 0.3 to about 5,000 parts by weight of the solvent be used for 1 part by weight of maca. In particular, 5 to 100 parts by weight of the solvent is used for 1 part by weight of maca to maximize the efficiency of the extraction process.

While the maca extracts obtained in the form of solution may be directly used as the peripheral blood flow-improving composition without further processing, the extracts may be concentrated by evaporating the solvent to make a concentrate, or the extracts may be dried by lyophilization or air-blowing to form a dry powder. The concentrated extracts and dry powders are preferred in terms of stability during storage and portability. The term “maca extracts” as used herein refers to any of these solutions, concentrates and dry powders.

The present invention provides a peripheral blood flow-improving composition containing the above-described maca extracts as an active ingredient, as well as food and beverage products, perfumes and cosmetics and pharmaceutical products containing the peripheral blood flow-improving composition. Maca has been eaten in Peru since ancient time and its extracts have been proven to be safe.

Another aspect of the present invention concerns pharmaceutical products, food and beverage products, perfumes and cosmetics that contain the peripheral blood flow-improving composition containing the maca extracts as an active ingredient, obtained as described above. The amount of the peripheral blood flow-improving composition in the pharmaceutical products, food and beverage products, perfumes and cosmetics may vary depending on the amount of maca extracts used in the peripheral blood flow-improving composition. The amount of maca extracts may be properly determined by considering such factors as the desired effect, the flavor and the color of the resulting products. The amount of maca extracts in the peripheral blood flow-improving composition (as measured by the dry weight) is typically from 0.01 to 99.9%, and preferably from 0.01 to 99.5%.

Examples of the food and beverage products provided by the present invention include candies, troches, chewing gums, yoghurt, ice cream, pudding, jelly, sweetened and jellied bean pastes (Mizu Yokan), alcohol beverages, coffee beverages, juice beverages, fruit juice beverages, sodas, soft drinks, milk, whey drinks, lactobacillus drinks and various other food and beverage products.

These food and beverage products can be prepared by conventional techniques and may be formulated with optional additives. Any additive commonly used in food products can be used in the preparation of these food and beverage products. Examples of such additives include glucose, fructose, sucrose, maltose, sorbitol, stevioside, rubsoside, corn syrup, lactose, citric acid, tartaric acid, malic acid, succinic acid, lactic acid, L-ascorbic acid, dl-α-tocopherol, sodium erythorbate, glycerol, propyleneglycol, glycerol fatty acid esters, polyglycerol fatty acid esters, sucrose fatty acid esters, sorbitan fatty acid esters, propylene glycol fatty acid esters, gum arabic, carrageenan, casein, gelatin, pectin, agar, vitamin Bs, nicotinamide, calcium pantothenate, amino acids, calcium salts, pigments, flavors and preservatives.

The term “perfumes and cosmetics” as used herein encompasses cosmetics and perfume products in general. Examples include non-oral products, such as face lotions, face creams, milky lotions, foundations, lipsticks, hairdressings, hair tonics, hair regrowth lotions, shampoos, rinses and bath agents; and oral products, such as toothpastes, mouthwashes, gargles and oral fragrances. The maca extracts in an appropriate amount may be added to these products to make desired cosmetic or perfume products.

These perfumes and cosmetics can be prepared by conventional techniques: any of the materials commonly used in perfumes and cosmetics may be added as desired. Examples of such materials include fats and oils, such as vegetable oils, waxes, such as lanoline and beeswax, hydrocarbons, fatty acids, higher alcohols, esters, surfactants, pigments, vitamins, plant and animal extracts, UV-absorbents, antioxidants, preservatives and bactericides.

The pharmaceutical products provided by the present invention may be formulated into various dosage forms by mixing the maca extracts with other optional additives. Examples of such dosage forms include oral preparations, such as tablets, capsules, granules, powders, syrups and extracts; and parenteral preparations, such as ointments, eye ointments, lotions, creams, patches, suppositories, nasal sprays and injections.

These pharmaceutical products may be formulated with various additives and can be prepared by conventional techniques. Any suitable additive according to Japanese Pharmacopoeia may be added, including solid carriers, such as starch, lactose, sucrose, mannitol, carboxymethylcellulose, corn starch and inorganic salts; liquid carriers, such as physiological saline, distilled water, glucose solution, alcohols such as ethanol, propylene glycol and polyethylene glycol; and oil carriers, such as animal and plant oils; white Vaseline, paraffins and waxes.

The maca extracts may be administered at any suitable daily dose. For example, the extracts may be administered at a daily dose of about 0.01 mg to about 10 g as measured by the dry weight. For oral administration, a daily dose of 1 mg to 1,000 mg (as measured by the dry weight) is preferable to expect particular improving the peripheral blood flow.

The peripheral blood flow-improving composition of the present invention is not only capable of improving the peripheral blood flow, but it is also safe for oral administration. For this reason, the composition is suitable for use in food and beverage products, oral pharmaceutical products and oral perfumes and cosmetics.

The maca extracts provided by the present invention may be used in conjunction with other peripheral blood flow-improving agents, such as vitamin E and capsaicin, to make food and beverage products, perfumes and cosmetics or pharmaceutical products.

As used herein, the term “additives or ingredients for use in food and beverage products” is intended to mean mixtures of additives or ingredients commonly used in food and beverage products, such as flavors, colors and antioxidants, with maca extracts. The proportions of additives or ingredients and maca extracts in the mixture are determined by the intended purpose of the products. The additives and ingredients may be those described above.

### EXAMPLES

The present invention will now be described with reference to examples and experiments. These examples, however, are provided by way of example only and are not intended to limit the scope of the invention in any way. Throughout the following examples, “%” means “% by weight” unless otherwise specified.

### Example 1

### Preparation of Maca Extracts

3 kg of a dried and crushed maca product were placed in a stainless steel vessel. To this vessel, 30 L of a 50% (by volume) aqueous ethanol solution were added and the solution was stirred for 3 hours at 60° C. for extraction. The solution was filtered and the solvent was removed from the filtrate to give 150 g of maca extract.

### Example 2

### Preparation of Maca Extracts

3 kg of a dried and crushed maca product were placed in a stainless steel vessel. To this vessel, 30 L of a 99% (by volume) aqueous ethanol solution were added and the solution was stirred for 3 hours at 60° C. The solution was filtered and the solvent was removed from the filtrate to give 180 g of maca extract.

### Example 3

### Pharmaceutical Products Containing Maca Extract

### (1) Tablets

Using a single stroke tablet machine, 66.7 g of the maca extract obtained in Example 1, 232.0 g of lactose and 0.5 g of magnesium stearate were made into 10 mm tablets each weighing 300 mg.

(2) Powders 0.5 g of magnesium stearate was added to 99.5 g of the maca extracts obtained in Example 1. The mixture was compressed, crushed, sorted and sieved to obtain 20 to 50 mesh granules.

### Example 4

### Food and Beverage Products Containing Maca Extract

Food and beverage products containing the maca extract obtained in Example 1 and having different compositions were prepared as shown below.

### (1) Candy

| (Composition) | (Parts by weight) |
|---|---|
| Sorbitol powder | 99.7 |
| Flavor | 0.2 |
| Maca extract | 0.05 |
| Sorbitol seed | 0.05 |
| Total | 100.00 |

### (2) Chewing Gum

| (Composition) | (Parts by weight) |
|---|---|
| Gum base | 20.0 |
| Calcium carbonate | 2.0 |
| Stevioside | 0.1 |
| Maca extract | 0.05 |
| Lactose | 76.85 |
| Flavor | 1.0 |
| Total | 100.00 |

### (3) Caramel

| (Composition) | (Parts by weight) |
|---|---|
| Granulated sugar | 32.0 |
| Starch syrup | 20.0 |
| Milk powder | 40.0 |
| Hardened oil | 4.0 |
| Table salt | 0.6 |
| Flavor | 0.02 |
| Water | 3.22 |
| Maca extract | 0.16 |
| Total | 100.00 |

### (4) Soda

| (Composition) | (Parts by weight) |
|---|---|
| Granulated sugar | 8.0 |
| Concentrated lemon juice | 1.0 |
| L-ascorbic acid | 0.10 |
| Citric acid | 0.09 |
| Sodium citrate | 0.05 |
| Coloring agent | 0.05 |
| Carbonated water | 90.70 |
| Maca extract | 0.01 |
| Total | 100.00 |

### (5) Juice Beverage

| (Composition) | (Parts by weight) |
|---|---|
| Frozen concentrated orange juice | 5.0 |
| High fructose corn syrup | 1.0 |
| Citric acid | 0.10 |
| L-ascorbic acid | 0.09 |
| Maca extract | 0.05 |
| Flavor | 0.20 |
| Coloring agent | 0.10 |
| Water | 93.46 |
| Total | 100.00 |

### (6) Lactobacillus Beverage

| (Composition) | (Parts by weight) |
|---|---|
| Fermented milk (Solid milk component = 21%) | 14.76 |
| High fructose corn syrup | 13.31 |
| Pectin | 0.50 |
| Citric acid | 0.08 |
| Flavor | 0.15 |
| Water | 71.14 |
| Maca extract | 0.06 |
| Total | 100.00 |

### (7) Alcohol Beverage

| (Composition) | (Parts by weight) |
|---|---|
| 50% ethanol | 32.0 |
| Sugar | 8.2 |
| Fruit juice | 2.4 |
| Maca extract | 0.4 |
| Water | 57.0 |
| Total | 100.0 |

### Example 5

### Perfumes and Cosmetics Containing Maca Extracts

Perfumes and cosmetics containing the maca extracts obtained in Example 1 and having different compositions were prepared as shown below.

### (1) Emollient Cream

| (Composition) | (Parts by weight) |
|---|---|
| Beeswax | 2.0 |
| Stearyl alcohol | 5.0 |
| Stearic acid | 8.0 |
| Squalane | 10.0 |
| Self-emulsifiable propylene glycol | 3.0 |
| Monostearate polyoxyethylene cetyl ether (20EO) | 1.0 |
| Perfume | 0.5 |
| Antioxidant | Trace |
| Preservative | Trace |
| Propylene glycol | 4.8 |
| Glycerol | 3.0 |
| Sodium hyaluronate | 0.1 |
| Maca extract | 0.1 |
| Triethanol amine | 1.0 |
| Purified water | 61.5 |
| Total | 100.0 |

### (2) Milky Foundation

| (Composition) | (Parts by weight) |
|---|---|
| Stearic acid | 2.4 |
| Propylene glycol monostearate | 2.0 |
| Cetostearyl alcohol | 0.2 |
| Liquid lanolin | 2.0 |
| Liquid paraffin | 3.0 |
| Isopropyl myristate | 8.5 |
| Propyl p-hydroxybenzoate | Trace |
| Purified water | 54.1 |
| Carboxymethylcellulose sodium | 0.2 |
| Bentonite | 0.5 |
| Propylene glycol | 3.8 |
| Sodium hyaluronate | 0.1 |
| Maca extract | 0.1 |
| Triethanol amine | 1.1 |
| Methyl p-hydroxybenzoate | Trace |
| Titanium oxide | 8.0 |
| Talc | 4.0 |
| Coloring content | Trace |
| Perfume | Trace |
| Squalane | 10.0 |
| Total | 100.0 |

### Experiment

### Ability of Maca Extract to Improve Peripheral Blood Flow

Healthy volunteered female subjects were tested for the effect of oral ingestion of a maca extract-containing alcohol beverage on the peripheral blood flow.

### (1) Subjects

10 females in 20's to 30's

### (2) Samples Tested

A “maca-containing liquor” which is maca-containing alcohol beverage prepared in (7) of Example 4 containing the maca extract obtained in Example 1 (50% alcohol extract) was tested. A “maca-free liquor” was used as a control that has the same composition as (7) of Example 4 except that the maca extract was excluded.

### (3) Method

10 subjects were randomly divided into two groups of five: one group was given the maca-containing liquor while the other group was given the maca-free liquor. Using crossover method, subjects were tested twice by at a one-week interval.

Each subject consumed 100 mL of the assigned sample and the peripheral blood flow was measured before and after the ingestion. 400 mg of maca extract were present in the 100 mL portion of maca-containing liquor.

### (4) Technique Used to Determine Improvement in the Peripheral Blood Flow

Using a digital laser blood flow meter (CyberMed CDF-2000, OAS Co., Ltd.), the blood flow was measured in the middle finger tip of each subject before and 60 minutes after the ingestion of the assigned sample.

### (5) Results

The results are shown in FIG. 1. Each data point indicates average of 10 subjects. For the maca-free liquor group, the peripheral blood flow did not change significantly between before and 60 minutes after the ingestion. In contrast, the peripheral blood flow for the maca-containing liquor group increased significantly 60 minutes after ingestion from the initial blood flow. After 60 minutes, the peripheral blood flow of the maca-containing liquor group was significantly (p<0.05) greater than that of the maca-free liquor group.

### INDUSTRIAL APPLICABILITY

As set forth, the peripheral blood flow-improving composition containing maca extracts of the present invention significantly improves peripheral blood flow and thus serves as an effective cure for cold sensation, numbness, pains and other symptoms associated with the disturbed peripheral blood flow. The peripheral blood flow-improving composition is safe and may be orally ingested to give the desired effects. The composition can ameliorate frostbites, chilblains, chaps and other skin problems caused by the disturbed peripheral blood flow and is thus of significant importance.

### BRIEF DESCRIPTION OF THE DRAWINGS

FIG. 1 is a diagram showing the results of Test Example of the present invention.

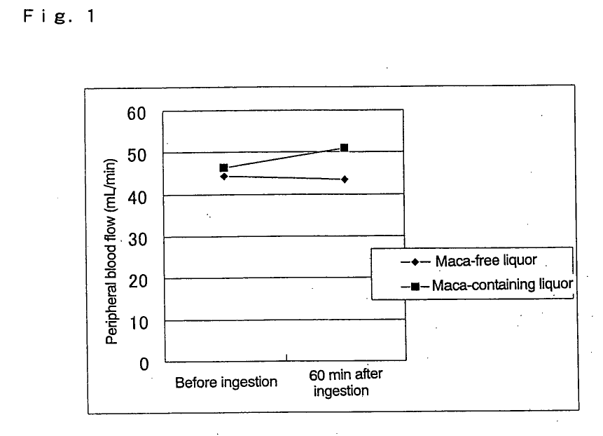

1-5. (canceled)

6. A method of improving cutaneous peripheral blood flow comprising administering an extract of a plant of the genus Lepidium of the family Cruciferae, as an active ingredient, to a human subject in need thereof to improve cutaneous peripheral blood flow.

7. The method for improving cutaneous peripheral blood flow according to claim 6, wherein the plant of the genus Lepidium of the family Cruciferae is maca (Lepidium meyenii Walp).

8. The method of improving cutaneous peripheral blood flow according to claim 6, wherein a maca extract is the active ingredient.

9. The method of improving cutaneous peripheral blood flow according to claim 8, wherein the maca extract is obtained by adding an aqueous ethanol solution to a crushed maca product to form a mixture, and by maintaining the mixture at 40° C. to 85° C. for extraction.

10. The method of improving cutaneous peripheral blood flow according to claim 6, wherein cutaneous peripheral blood flow is digital peripheral blood flow.

11. The method of improving cutaneous peripheral blood flow according to claim 10, wherein digital peripheral blood flow is peripheral blood flow of middle finger tip.

12. A method of improving skin disorders comprising administering an extract of a plant of the genus Lepidium of the family Cruciferae, as an active ingredient, to a human subject in need thereof to improve skin disorders caused by disturbed peripheral blood flow.

13. The method of improving skin disorders according to claim 12, wherein skin disorders are frostbites, chilblains, or chaps.
